(19) United States
(12) Patent Application Publication
(10) Pub. No.: US 2026/0274899 A1
(43) Pub. Date: Sep. 17, 2026

(54) METHODS FOR EXTRACTING AND ENCAPSULATING PHYCOCYANIN

(71) Applicant: ARIZONA BOARD OF REGENTS ON BEHALF OF ARIZONA STATE UNIVERSITY, Scottsdale, AZ (US)

(72) Inventors: Willem Frederik Johan VERMAAS, Tempe, AZ (US); Sunil TIWARI, Tempe, AZ (US)

(21) Appl. No.: 19/168,776

(22) PCT Filed: Mar. 27, 2024
(86) PCT No.: PCT/US2024/021621

§ 371 Date: Sep. 24, 2025

Related U.S. Application Data
(60) Provisional application No. 63/492,552, filed on Mar. 28, 2023.

(51) Int. Cl. C07K 14/195; C07K 1/30; C12N 1/20; C12R 1/01

(52) U.S. Cl. CPC C07K 14/195; C07K 1/30; C12N 1/20; C12R 2001/01

(57) ABSTRACT

Provided herein are methods for the isolation of phycocyanin from cells and purification. Phycocyanin is encapsulated and protected from exposure to water, enhancing the thermostability. The thermostability conferred by the encapsulation as described herein improves the use of phycocyanin in pigment compositions and food coloring as a replacement of synthetic blue dyes.

### CROSS-REFERENCE TO RELATED APPLICATIONS

This application claims priority to U.S. Provisional Patent Application No. 63/492,552, filed Mar. 28, 2023, the content of which is hereby incorporated by reference in its entirety.

### STATEMENT REGARDING FEDERALLY SPONSORED RESEARCH

This invention was made with government support under DE-EE0009274 awarded by the U.S. Department of Energy. The government has certain rights in the invention.

### BACKGROUND

Phycocyanin is a bright blue pigment-protein complex found in cyanobacteria. The phycocyanin complex is a light-harvesting phycobiliprotein that aggregates to form clusters, or phycobilisomes, that adhere to cellular membranes, making the isolation of phycocyanin challenging. Phycocyanin makes up a large fraction (as much as 25%) of the dry weight of cyanobacterial cells.

Phycocyanin has industrial applications including food coloring, nutraceuticals, and cosmetics. The phycocyanin market is growing with the demand for food products that are free of synthetic chemicals and public preference towards natural over synthetic pigments. Phycocyanin is also relevant to biological research and medicine as phycocyanin has antioxidant, anticancer, antiviral, and anti-inflammatory properties. However, phycocyanin is difficult to extract from biomass and readily denatures at high temperatures. Obstacles in commercial production and use of phycocyanin are the high cost of purification and the relatively low stability of phycocyanin when exposed to high temperatures (for example, greater than 45° C.).

The current phycocyanin market is dominated by phycocyanin isolated from outdoor cultures of Arthrospira platensis (syn. Spirulina platensis). General purification steps of crude phycocyanin include mechanical or chemical extraction, precipitation, chromatography, and dialysis. However, these techniques are not readily scalable. Even when sufficiently pure phycocyanin has been isolated, it readily denatures at 45-70° C., depending on the strain of cyanobacteria or algae from which the phycocyanin originated. This temperature range is too low for many of the potential applications (food coloring, etc.) of phycocyanin.

There remains a need for an improved procedure for the isolation of phycocyanin that can be scaled to industrially relevant amounts and with high purity. Further, there remains a need to stabilize phycocyanin to thermal conditions that may be encountered in industrial applications.

### SUMMARY

In one aspect, the present disclosure provides a method of extracting phycocyanin, the method comprising adding a salt of a divalent metal ion to a slurry comprising phycocyanin to a final concentration of the salt of about 0.25% w/v to about 1% w/v, homogenizing the slurry to form a homogenate, centrifuging the homogenate to form a pellet and a supernatant comprising phycocyanin, and isolating phycocyanin from the supernatant to form a phycocyanin extract.

In some embodiments, the divalent metal ion is Ca²⁺. For example, the salt of the divalent metal ion can be calcium chloride (CaCl₂)). In some embodiments, isolating phycocyanin from the supernatant comprises: isolating the supernatant from the pellet, combining the isolated supernatant with ammonium sulfate to induce precipitation of the phycocyanin, and isolating the precipitated phycocyanin to form the phycocyanin extract.

In some embodiments, phycocyanin can be extracted from a cyanobacterial biomass. The cyanobacterial biomass can be obtained, for example, from cyanobacterial cultures of Synechocystis sp. PCC 6803, Spirulina platensis UTEX LB2340, Synechococcus sp. PCC 11901, Synechococcus sp. PCC 7002, or Thermosynechococcus vestitus BP-1. In some embodiments, the method of extracting phycocyanin further comprises drying the phycocyanin extract.

In another aspect, the present disclosure provides a phycocyanin extract produced by the extraction method as described herein, including a dried phycocyanin extract produced by the present extraction method. The dried phycocyanin extract can be, for example, a powder. The ratio of the absorbance at 620 nm to the absorbance at 280 nm (A₆₂₀/A₂₈₀ ratio) for the dried phycocyanin extract can be at least 0.7 (food grade) or at least 4 (analytical grade).

In another aspect, the present disclosure provides a method of encapsulating phycocyanin, which comprises contacting a first liquid phase comprising phycocyanin and a polar solvent with an encapsulating agent comprising a lipid to form an encapsulation mixture, thereby the phycocyanin is encapsulated in the encapsulating agent. The first liquid phase can be essentially free of water. In some embodiments, the method comprises combining the first liquid phase with a second liquid phase comprising the encapsulating agent and a nonpolar solvent to form the encapsulation mixture, thereby the phycocyanin is encapsulated in the encapsulating agent. The first liquid phase and the second liquid phase can be essentially free of water.

In some embodiments, the first liquid phase is a suspension or solution prepared by mixing dry phycocyanin with the polar solvent. In some embodiments, the second liquid phase is a solution of the encapsulating agent in the nonpolar solvent. In some embodiments, the method further comprises removing the polar solvent and the nonpolar solvent to form a dried encapsulation mixture. In some embodiments, the method further comprises resuspending the dried encapsulation mixture in a resuspending buffer to form a resuspended encapsulation mixture. In some embodiments, the method further comprises isolating encapsulated phycocyanin from the resuspended encapsulation mixture by precipitation, thereby producing an encapsulated phycocyanin product. In some embodiments, the method further comprises drying the encapsulated phycocyanin product.

In some embodiments, the encapsulating agent comprises a phospholipid, a di-ether lipid, a tetra-ether lipid, cholesterol, or a mixture thereof. The phospholipid can include, for example, phosphatidylcholine (PC), phosphatidylethanolamine (PE), sphingomyelin (SM), or a mixture thereof. The encapsulating agent can further comprise a protein, such as a membrane protein. In some embodiments, the encapsulating agent comprises: (a) milk fat globule membranes (MFGM), (b) phosphatidylcholine and cholesterol; (c) a diether lipid; (d) a tetra-ether lipid; or (e) a mixture thereof. In some embodiments, the encapsulation agent comprises milk fat globule membranes, such as the milk fat globule membranes derived from cow milk.

In some embodiments, the polar solvent comprises methanol, acetone, tetrahydrofuran, dimethylformamide, acetonitrile, dichloromethane, or a mixture thereof. In some embodiments, the nonpolar solvent comprises ethyl acetate, methyl t-butyl ether, cyclohexane, benzyl benzoate, hexanes, or a mixture thereof.

In some embodiments, prior to encapsulation, the phycocyanin is treated to have a net positive surface charge and the encapsulating agent is treated to have a net negative surface charge. In some embodiments, the first liquid phase further comprises a surfactant.

In some embodiments, the present disclosure provides an encapsulated phycocyanin product produced by the encapsulation method as described herein, such as a dried encapsulated phycocyanin product produced by the encapsulation method as described herein.

In another aspect, the present disclosure provides a pigment composition comprising phycocyanin encapsulated in an encapsulating agent, the encapsulating agent comprising a lipid. For example, the encapsulating agent in the pigment composition can comprise milk fat globule membranes, a di-ether lipid, a tetra-ether lipid, cholesterol, a phospholipid, a glycolipid, a glycoprotein, a carbohydrate, or a mixture thereof. In some embodiments, the encapsulated phycocyanin maintains color after a heat treatment of 100° C. for at least 5 min, as determined by a change in the absorbance at 620 nm of less than 20%.

In yet another aspect, the present disclosure provides a food colorant comprising phycocyanin and milk fat globule membranes, wherein the phycocyanin is encapsulated in the milk fat globule membranes.

In some embodiments, the present disclosure provides a pigment composition or a food colorant comprising the encapsulated phycocyanin product produced by method described herein.

### BRIEF DESCRIPTION OF THE DRAWINGS

FIG. 1 shows a plot of the change in absorbance at 620 nm over 24 hours for dry phycocyanin (100° C., “Dry”) and for an aqueous solution of phycocyanin (50° C., “Wet”).

FIG. 2 shows agarose-gel electrophoresis of phycocyanin at different pH conditions. The pH of the phycocyanin solution and sodium acetate buffer running buffer was at 5.0 (panel A), 4.8 (panel B) and 4.5 (panel C). Samples were loaded in the sample loading well, and migration in the electric field was followed.

FIG. 3 shows agarose-gel electrophoresis of MFGM samples at pH 9, pH 6.5, pH 5, and pH 4.2. Samples were loaded in the sample loading well, and migration in the electric field within the loading well was followed.

FIGS. 4A-4C show absorbance spectra of control (FIG. 4A), encapsulated phycocyanin (FIG. 4B, sample M5 in Table 3), and freshly isolated phycocyanin (FIG. 4C) before and after boiling at 100° C. for time periods of 0 min (“before”), 5 min, 10 min, and 15 min. Spectra have been corrected for scattering.

### DETAILED DESCRIPTION

The following discussion is presented to enable a person skilled in the art to make and use embodiments of the invention. Various modifications to the illustrated embodiments will be readily apparent to those skilled in the art after having studied the teachings in this disclosure, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans, after having studied the teachings in this disclosure, will recognize the examples provided herein have many useful alternatives and fall within the scope of embodiments of the invention.

Phycocyanin is a commercially desirable natural pigment that is bright blue, having an absorption maximum around 620 nm. Phycocyanin is known to make up as much as 25% of the dry weight of cyanobacterial cells, making it an attractive target for extraction from cultured cells. While phycocyanin is an ideal candidate for food coloring, cosmetics and other applications, previous extraction techniques are not efficient. Furthermore, phycocyanin is known to readily denature when exposed to heat, which can limit the utility and application of phycocyanin.

The present disclosure provides a simple, scalable method for phycocyanin extraction and purification. The present extraction method can be used for extracting phycocyanin from various cyanobacterial strains.

Further, present disclosure provides a simple, scalable method for phycocyanin encapsulation. Furthermore, the present disclosure includes a process that results in dry phycocyanin product, which is remarkably thermostable. Thus, the present disclosure provides a simple phycocyanin extraction procedure and an encapsulation procedure that yields thermostable phycocyanin.

In various embodiments, the present disclosure demonstrates that phycocyanin can be suspended in some organic solvents while largely retaining its absorbance characteristics and that encapsulating this phycocyanin in natural extracts, all in the absence of water, yields phycocyanin that is highly thermostable and can even be boiled. Thus, the methods and processes disclosed herein provide an efficient, industrially viable approach to extract and purify phycocyanin at high yield and purity.

### Phycocyanin Extraction

In one aspect, the present disclosure provides a method of extracting phycocyanin, the method comprising:
adding a salt of a divalent metal ion to a slurry comprising phycocyanin to a final concentration of the salt of about 0.25% w/v to about 1% w/v;
homogenizing the slurry to form a homogenate;
centrifuging the homogenate to form a pellet and a supernatant comprising phycocyanin; and
isolating phycocyanin from the supernatant to form a phycocyanin extract.

The slurry can be an aqueous suspension that includes phycocyanin or a source of phycocyanin, such as a cyanobacterial biomass that contains phycocyanin. The cyanobacterial biomass can include, for example, intact or broken cells of various cyanobacterial strains. Suitable cyanobacterial strains used as phycocyanin sources herein can include a variety of seaweeds and cyanobacterial species, such as Aphanizomenon sp., Spirulina (Arthrospira) sp., Phormidium sp., Lyngbya sp., Synechocystis sp., and Synechococcus sp. (Jiang, et al. J Cancer. 2017; 8 (17): 3416-3429. Doi: 10.7150/jca.21058). In some embodiments, the slurry includes cyanobacterial biomass derived from Synechocystis sp. PCC 6803, Spirulina platensis UTEX LB2340 (also known as Arthrospira platensis UTEX LB2340), Synechococcus sp. PCC 11901, Synechococcus sp. PCC 7002, Thermosynechococcus vestitus BP-1, or a combination thereof.

The cyanobacterial strains can be cultured and harvested by known techniques. In some embodiments, cultures of these phycocyanin-producing strains of cyanobacteria can be grown in an appropriate environment with media chosen to support growth of a cyanobacterial biomass comprising the cells containing the phycocyanin. The environment can include outdoor environments or climate-controlled indoor environments, the media can include wastewater or grey water, fresh, brackish or salt water, minerals needed for growth of the organisms, aeration or other forms of providing CO₂ for growth, and light exposure to allow photosynthesis to occur.

In some embodiments, the cyanobacterial cultures are grown until the optical density of the culture at 730 nm (OD 730; a measure of culture density) reaches a value of about 4. The culture can be left undisturbed in a dark environment to allow the biomass to settle in a container. The culture can be left in the dark for 18 hours, for 24 hours, or for 36 hours to settle. Once the culture has settled, a portion of the clear culture medium can be decanted away to obtain a slurry comprising cyanobacterial biomass. The portion of the culture medium that is decanted can be about 60%, about 70%, about 80%, about 90%, or greater than 90% of the original volume.

The extraction can be carried out in the presence of a divalent metal ion added to the slurry. For example, a salt of the divalent metal ion can be added to the slurry. The salt in general is soluble in the slurry. For example, the slurry can be an aqueous slurry and the salt of the divalent metal ion is soluble in water. The salt can also be stable in the slurry and during the extraction process (e.g., resistant to oxidation and without precipitation). In some embodiments, the divalent metal ion includes Ca²⁺, Mg²⁺, Zn²⁺, or a combination thereof. In some embodiments, the salt of the divalent metal ion includes chloride, bromide, nitrate, acetate, sulfate, or a combination thereof. In some embodiments, the salt of the divalent metal ion includes calcium chloride (CaCl₂)). In some embodiments, the salt of the divalent metal ion is calcium chloride.

In some embodiments, the salt can be added to the slurry to a final concentration of about 0.25% w/v to about 1% w/v, including but not limited to about 0.25% w/v to about 0.8% w/v, about 0.25% w/v to about 0.7% w/v, about 0.3% w/v to about 0.7% w/v, and about 0.4% w/v to about 0.6% w/v. The salt concentration in the slurry can be about 0.25% w/v, about 0.5% w/v, about 0.75%, and about 1% w/v. In some embodiments, calcium chloride is added to the slurry to a final concentration of about 0.25% w/v to about 1% w/v, such as about 0.3% w/v to about 0.7% w/v. In some embodiments, calcium chloride is added to the slurry to a final concentration of about 0.5% w/v.

The slurry with the salt (e.g., CaCl₂)) can be homogenized to break open cells and form a homogenate. The homogenization can be accomplished, for example, using means such as ultrasonication, high-pressure methods, electric pulses, bead-beating using glass, ceramic, or steel beads, or even freezing and thawing, depending on the strain of cyanobacteria used. In some embodiments, the homogenization is carried out using a bead beater. For example, a bead beater can be used at 4 cycles of 45 s each in presence of an equal volume of 0.1 mm glass beads.

The homogenate (e.g., including the broken cells) can be then centrifuged to separate the phycocyanin, which localizes in the liquid supernatant, from the solid components, which form a pellet. In some embodiments, the centrifugation is carried out at a predetermined speed (e.g., at least 12,000×g) and temperature (e.g., 4° C.). For example, the homogenate can be centrifuged at 12,000×g for 10 min at 4° C. The supernatant containing the phycocyanin can be separated from the solid pellet by pipetting or decanting.

In some embodiments, isolating phycocyanin from the supernatant can include:
isolating the supernatant from the pellet,
combining the isolated supernatant with ammonium sulfate to induce precipitation of the phycocyanin, and
isolating the precipitated phycocyanin to form the phycocyanin extract.

To induce precipitation of phycocyanin from the isolated supernatant, ammonium sulfate can be added slowly to the isolated supernatant with stirring to form a solid precipitate comprising the phycocyanin, or a phycocyanin extract. The ammonium sulfate can be added to the isolated supernatant up to a concentration of about 277 g/L (w/v). In some embodiments, the ammonium sulfate can be added up to at about 40% saturation, about 45% saturation, about 50% saturation, about 55% saturation, about 60% saturation, about 65% saturation, or about 70% saturation. In some embodiments, the ammonium sulfate can be added up to about 45% saturation at 25° C. Once the addition of the ammonium sulfate is complete, the phycocyanin extract can be separated by centrifugation, for example at 4° C., 12,000×g for 15 min. Centrifugation of the resulting mixture provides an ammonium sulfate supernatant and the phycocyanin extract as a solid. The phycocyanin extract can be isolated as a solid by filtration, pipetting, or decanting away the ammonium sulfate supernatant.

The pellet resulting from centrifugation of the homogenate (e.g., prior to ammonium sulfate precipitation) can include remaining phycocyanin, or a small amount of phycocyanin that is captured within the pellet during the centrifugation process. To increase recovery of phycocyanin, the centrifugation process can be repeated one or more times. In some embodiments, isolating the supernatant (containing phycocyanin) from the pellet can further include:
resuspending the pellet in about 0.25% w/v to about 1% w/v CaCl₂) to form a suspension;
centrifuging the suspension to form an additional supernatant comprising phycocyanin; and
combining the supernatant with the additional supernatant to form the isolated supernatant.

For example, the pellet can be resuspended using 0.5% w/v CaCl₂) in water to form a suspension. This step can release remaining phycocyanin from the pellet as phycocyanin is water soluble. An additional centrifugation of the suspension forms an additional supernatant. Following this repeated process, the additional supernatant resulting from each step can be combined with the supernatant to form an isolated supernatant. The isolated supernatant can be centrifuged to remove any remaining particulate matter.

In some embodiments, the phycocyanin extract can be dissolved in water and the dissolved phycocyanin extract can be subject to a second centrifugation step, for example at 4° C., 12,000×g for 15 min, resulting in a phycocyanin supernatant. In this step, the water used for dissolving the phycocyanin does not have ammonium sulfate and thus the phycocyanin does not precipitate in this step. This second centrifugation step can separate remaining cell debris and contaminants from the phycocyanin supernatant. The phycocyanin remains dissolved in the phycocyanin supernatant as the ammonium sulfate concentration has decreased. In some embodiments, the solvent can be removed from the phycocyanin supernatant to yield a dried phycocyanin extract.

In some embodiments, the phycocyanin supernatant, or the redissolved phycocyanin extract, is subject to dialysis against water or ultrafiltration methods to separate salts or other low molecular weight species from the phycocyanin. The dialysis or ultrafiltration step followed by drying can result in a purified phycocyanin extract.

The purity and quality of the phycocyanin extract can be characterized by the ratio of the absorbance at 620 nm (A₆₂₀) to the absorbance at 280 nm (A₂₈₀). For example, if this ratio is 0.7 or greater, the phycocyanin is regarded as food grade. If this ratio is 4 or greater, the phycocyanin is regarded as analytical grade. These guidelines for the A₆₂₀/A₂₈₀ ratio can be found, for example, in Rito-Palomares et al. (“Practical application of aqueous two-phase systems for the development of a prototype process for C-phycocyanin recovery from Spirulina maxima”, J. Chem. Technol. Biotechnol. 76 (2001) 1273-1280). In some embodiments, the present extraction method can produce an isolated supernatant containing phycocyanin with an A₆₂₀/A₂₈₀ ratio of 1.5, which exceeds the criterion for the phycocyanin to be considered food-grade purity. The isolated supernatant prepared in this manner can be stored at 4° C. Additionally, and alternatively, the isolated supernatant can be dried by removing the solvent to produce a crude extract. In some embodiments, the extracted phycocyanin (e.g., in the supernatant from the cyanobacterial biomass or in the redissolved phycocyanin extract) is subject to dialysis or ultrafiltration to further improve the purity of the phycocyanin extract. In some embodiments, the A₆₂₀/A₂₈₀ ratio of the purified phycocyanin extract can be 4 or greater, corresponding to an analytical grade of purity. In some embodiment, the present method can provide a phycocyanin extract having an A₆₂₀/A₂₈₀ ratio of 4 or greater without requiring non-scalable techniques.

In some embodiments, the present method further comprises drying the phycocyanin extract. In some embodiments, the phycocyanin extract can be dried using a spray dryer. The spray dryer can have an inlet temperature of about 150° C. to about 180° C., The temperature can be, for example, about 150° C., about 160° C., about 170° C., or about 180° C. In other embodiments, the phycocyanin solution can be dried using a freeze dryer. The dried phycocyanin can be ground using mortar and pestle or using a Cryo-mill. The phycocyanin particle size thus obtained can be less than 10 μm, such as less than 5 μm, or less than 1 μm, or less than 0.5 μm. In some embodiments, the dried phycocyanin extract can be dispersed or dissolved again (e.g., in water) for further purification, and the further purified phycocyanin extract can be dried again by similar drying methods.

The amount of phycocyanin isolated by the present disclosure can be about 80%, about 85%, about 90% or about 95% as compared to the amount of phycocyanin originally present in the cells. The present disclosure demonstrates that this simple purification process is successful for all four strains of cyanobacteria that were tested (Synechocystis sp. PCC 6803, Spirulina platensis UTEX LB2340 (also known as Arthrospira platensis UTEX LB2340), Synechococcus sp. PCC 7002, and Thermosynechococcus vestitus BP-1), yielding a phycocyanin recovery rate of at least 90%. That the results were similar for all four strains of cyanobacteria suggests that this procedure may be applicable to other strains of cyanobacteria as well. Thus, a simple phycocyanin extraction and purification method is developed, which is cost effective and can work for a spectrum of cyanobacterial strains.

In another aspect, the present disclosure provides a phycocyanin extract produced by the extraction method as described herein.

In another aspect, the present disclosure provides a dried phycocyanin extract produced by the extraction method as described herein. The phycocyanin extract can be dried, for example, by spray drying or freeze drying. In some embodiments, the dried phycocyanin extract is a solid. In some embodiments, the dried phycocyanin extract is a powder. The dried phycocyanin extract can have a A₆₂₀/A₂₈₀ ratio of at least 0.7, including but not limited to at least 1, at least 1.2, at least 1.5, at least 2, at least 2.5, at least 3, at least 3.5, and at least 4. In some embodiments, the dried phycocyanin extract has a A₆₂₀/A₂₈₀ ratio of at least 1.5. In some embodiments, the dried phycocyanin extract has a A₆₂₀/A₂₈₀ ratio of at least 4.

The use of calcium chloride in phycocyanin isolation allows for facile isolation of substantially pure phycocyanin, far exceeding the requirements for food-grade phycocyanin. Moreover, with only one additional step of purification (e.g., precipitation with 45% ammonium sulfate) analytical grade phycocyanin can be obtained. This purification can be completed within a day, can be performed at room temperature on a large scale and does not require any specialized techniques, thus greatly reducing the cost of purification.

### Phycocyanin Encapsulation

In another aspect, the present disclosure provides a method of encapsulating phycocyanin, the method comprising:
contacting a first liquid phase comprising phycocyanin and a polar solvent with an encapsulating agent comprising a lipid to form an encapsulation mixture, thereby the phycocyanin is encapsulated in the encapsulating agent,
wherein the first liquid phase is essentially free of water.

The present method can be used for encapsulating dry phycocyanin. The dry phycocyanin can include, for example, a dried phycocyanin extract produced by the extraction method as described herein. The first liquid phase can be a suspension or a solution of the phycocyanin in the polar solvent. In some embodiments, the first liquid phase is a suspension or solution prepared by mixing dry phycocyanin with the polar solvent. The first liquid phase can be prepared, for example, by mixing a powdered phycocyanin with the polar solvent. The dry or powdered phycocyanin can have a A₆₂₀/A₂₈₀ ratio of at least 0.7, including but not limited to a A₆₂₀/A₂₈₀ ratio of at least 1, at least 1.5, at least 2, at least 2.5, at least 3, at least 3.5, or at least 4. The dry or powdered phycocyanin can include the dried phycocyanin extract or the purified phycocyanin prepared by the methods described herein. The dry or powdered phycocyanin can contain at least 10%, at least 15%, at least 20%, at least 25%, at least 35%, at least 40%, at least 50%, at least 60%, at least 70%, at least 80%, at least 85%, at least 90%, at least 95%, or at least 99% by weight of phycocyanin.

In some embodiments, prior to encapsulation, the phycocyanin is treated to have a net positive surface charge. For example, the first liquid phase can be prepared by mixing dry phycocyanin with the polar solvent, and the dry phycocyanin is prepared from drying a suspension of phycocyanin having a pH of about 4.5 to about 4.8. As nonlimiting examples, dry phycocyanin prepared from drying a suspension of phycocyanin having a pH of about 4.5 to about 4.8 can result in phycocyanin having a net positive surface charge.

The polar solvent can be, for example, methanol, acetone, tetrahydrofuran, dimethylformamide, acetonitrile, dichloromethane, or a mixture thereof. The polar solvent or the first liquid phase can be essentially free of water. The term “essentially free of water” refers to less than 1%, less than 0.5%, less than 0.1%, or less than 0.05% water. In some embodiments, the polar solvent or the first liquid phase is free of water.

In some embodiments, the first liquid phase further comprises a surfactant. The surfactant can facilitate the solubility or dispersion of phycocyanin (e.g., dried phycocyanin extract produced by the extraction method as described herein). The surfactant can be a mild surfactant, including a nonionic surfactant. The surfactant can be an ester-based nonionic surfactant. In some embodiments, the surfactant is a fatty acid ester, such as a sugar ester of a fatty acid, including but not limited to, sugar esters of lauric acid, myristic acid, palmitic acid, stearic acid, oleic acid, behenic acid, and combinations thereof. In some embodiments, the surfactant is sucrose laurate.

The first liquid phase may be allowed to directly contact the encapsulating agent. Alternatively, the encapsulating agent can be dissolved or suspended in another medium before being introduced to the first liquid phase. In some embodiments, the present method comprises:
combining the first liquid phase with a second liquid phase comprising the encapsulating agent and a nonpolar solvent to form the encapsulation mixture, thereby the phycocyanin is encapsulated in the encapsulating agent,
wherein the first liquid phase and the second liquid phase are essentially free of water.

In some embodiments, the method comprises mixing the first liquid phase and the second liquid phase to form a mixture and agitating the mixture, thereby forming the encapsulating mixture.

In some embodiments, the second liquid phase is a solution of the encapsulating agent in the nonpolar solvent. The nonpolar solvent can be, for example, ethyl acetate, methyl t-butyl ether, cyclohexane, hexanes, benzyl benzoate, or a mixture thereof. The nonpolar solvent or the second liquid phase can be essentially free of water. In some embodiments, the nonpolar solvent or the second liquid phase is free of water. In some embodiments, the polar solvent and the nonpolar solvent are miscible. In some embodiments, the polar solvent and the nonpolar solvent are immiscible.

Encapsulation of phycocyanin can include formation of a complex between phycocyanin and an encapsulating agent. The encapsulating agent can include, for example, a lipid, an amphiphile, a membrane protein, an albumin, a peptide amphiphile, a lipid membrane, or a combination thereof. In some embodiments, the encapsulating agent includes a phospholipid, a glycolipid, a di-ether lipid, a tetra-ether lipid, cholesterol, or a mixture thereof. In some embodiments, the encapsulating agent includes a lipid that is from hyperthermophilic archaea and bacteria. In some embodiments, the encapsulating agent includes a phospholipid. Suitable phospholipids include, but are not limited to phosphatidylcholine (PC), phosphatidylethanolamine (PE), sphingomyelin (SM), and a mixture thereof.

In some embodiments, the encapsulating agent further includes a protein. In some embodiments, the encapsulating agent further includes a membrane protein. In some embodiments, the encapsulating agent comprises (a) milk fat globule membranes (MFGMs), (b) phosphatidylcholine and cholesterol; (c) a diether lipid; (d) a tetra-ether lipid; or (e) a mixture thereof.

In some embodiments, the encapsulating agent includes milk fat globule membranes (MFGMs). Milk fat globule membranes are structurally complex milk components composed of various lipids and proteins that surround milk fat globules. MFGM-derived fractions contain different classes of phospholipids including zwitterionic phosphatidylcholine (PC), phosphatidylethanolamine (PE), and sphingomyelin (SM). Each of these phospholipids constitutes between about 20% and 40% of the lipids in the MFGM. MFGM-derived fractions have more saturated phospholipids than their soy counterparts and thus have a higher phase transition temperature (Tₘ) that leads to higher thermostability. They also have glycosylated proteins, which can confer increased liposome integrity and an increased phase transition temperature. MFGMs can be isolated from fresh cow milk according to Wang et al. (Advancement on Milk Fat Globule Membrane: Separation, Identification, and Functional Properties. Frontiers in Nutrition, vol. 8, 2022, doi=10.3389/fnut.2021.807284.), which is incorporated herein by reference in its entirety.

In some embodiments, the encapsulating agent includes milk fat globule membranes, which are derived from cow milk. In some embodiments, the encapsulating agent includes phosphatidylcholine and cholesterol.

In some embodiments, prior to encapsulation, the encapsulating agent is treated to have a net negative surface charge. For example, the encapsulating agent can include milk fat globule membranes, which is prepared from drying a suspension of the milk fat globule membranes having a pH of about 6.5. As nonlimiting examples, milk fat globule membranes prepared from drying a suspension of the milk fat globule membranes having a pH of about 6.5 can result in milk fat globule membranes having a net negative surface charge.

Combining the first liquid phase and the second liquid phase as disclosed herein form an encapsulation mixture, thereby the phycocyanin is encapsulated in the encapsulating agent. In the encapsulation mixture, the phycocyanin may be in contact with the encapsulating agent. In some embodiments, there is no water near the encapsulated phycocyanin molecules. The method of encapsulation can further include agitating the mixture by shaking, vortexing, or stirring the mixture, and thereby the phycocyanin is encapsulated by the encapsulating agent. In some embodiments, the encapsulation of phycocyanin can include formation of a membrane-bounded species such as a vesicle, a micelle, or a liposome. The membrane-bounded species can include a monolayer membrane, a bilayer membrane, a trilayer membrane, or a multilayer membrane. The membrane-bounded species can be a lipid droplet where the phycocyanin is embedded within the lipid droplet. The membrane-bounded species need not have an intact, continuous monolayer or bilayer. The phycocyanin can be complexed with the membrane-bounded species to form the encapsulated phycocyanin.

Solvents can be removed from the encapsulation mixture to form a dried encapsulation mixture. In some embodiments, the method further comprises removing the polar solvent and the nonpolar solvent to form a dried encapsulation mixture. In some embodiments, the method further comprises redissolving or resuspending the dried encapsulation mixture in a resuspension buffer to form a resuspended encapsulation mixture, which includes the encapsulated phycocyanin. The resuspension buffer can have a pH of about 3 to about 11, including but not limited to, about 4 to about 10, about 5 to about 9, about 6 to about 8, and about 7 to about 8. In some embodiments, the resuspension buffer is a phosphate buffer with a pH of about 7.5.

In some embodiments, the method further comprises isolating encapsulated phycocyanin from the resuspended encapsulation mixture, for example, by precipitation, thereby producing an encapsulated phycocyanin product. In some embodiments, the encapsulated phycocyanin product can be dried (e.g., in vacuo) to produce a dry encapsulated phycocyanin product. In dried form, the encapsulated phycocyanin product can have a stability suitable for long term storage, shipping and applications where a solid form is useful. In water, the encapsulated phycocyanin can be stable as well. In some embodiments, the encapsulated phycocyanin product has a A₆₂₀/A₂₈₀ ratio of at least 0.7 (corresponding to food grade), at least 1, at least 1.5, at least 2, at least 2.5, at least 3, at least 3.5, or at least 4 (corresponding to analytical grade).

In another aspect, the present disclosure provides an encapsulated phycocyanin product produced by the method as described herein.

In another aspect, the present disclosure provides a dried encapsulated phycocyanin product produced by the method as described herein.

Thermostability of phycocyanin is an important factor for widespread use in food and other applications. The thermotolerance of phycocyanin can be reported as A₆₂₀, the absorbance at 620 nm, as a function of heat treatment in terms of time and temperature. A₆₂₀ captures the deep-blue color of the phycocyanin and is indicative of the status of the phycocyanin. For example, phycocyanin in aqueous solution loses its blue color quickly when heated above 50° C. due to degradation. This thermal degradation is among the main reasons why phycocyanin has not been used at large scale as a food colorant that is a safe alternative to chemical dyes.

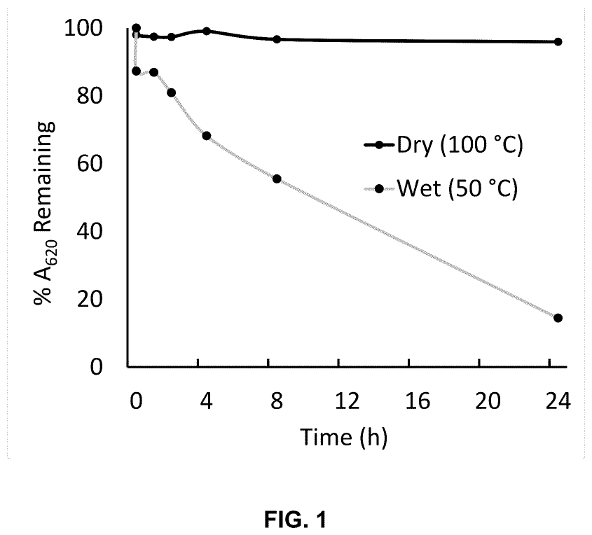

Surprisingly, as shown in FIG. 1, the present disclosure demonstrates that only a small decrease in A₆₂₀ is observed for dry phycocyanin when exposed to 100° C. for several hours. The present disclosure further demonstrates that phycocyanin can be stabilized against heat in the presence of water by encapsulation. Advantageously, the encapsulation of phycocyanin in lipids in organic solvents as described herein provides a relatively simple yet highly effective solution to protect phycocyanin from thermal degradation, including thermal degradation in water.

The method of phycocyanin encapsulation and purification as disclosed herein is simple, reducing the cost and time of phycocyanin production from a wide range of cyanobacteria. The thermostability conferred by the encapsulated phycocyanin opens new avenues of application. This phycocyanin (e.g., the encapsulated phycocyanin product as described herein) can be used as food coloring and can replace the currently used synthetic blue dyes that may have health impacts. Together, these developments allow for serious consideration of phycocyanin as a natural replacement of synthetic blue dyes in the food industry and in other applications.

### Food Colorant and Pigment Compositions

In another aspect, the present disclosure provides a pigment composition comprising phycocyanin encapsulated in an encapsulating agent, the encapsulating agent comprising a lipid. In some embodiments, the pigment composition includes an encapsulated phycocyanin product produced by the method as described herein. In some embodiments, the encapsulating agent includes milk fat globule membranes, a di-ether lipid, a tetra-ether lipid, cholesterol, a phospholipid, a glycolipid, a glycoprotein, a carbohydrate, or a mixture thereof. The phospholipid can include, for example, phosphatidylcholine (PC), phosphatidylethanolamine (PE), sphingomyelin (SM), or a mixture thereof.

The encapsulated phycocyanin in the pigment composition can have remarkable thermostability. In some embodiments, the encapsulated phycocyanin maintains color after a heat treatment of 100° C. for at least 5 min, as determined by a change in the absorbance at 620 nm of less than 20%, including less than 15%, less than 10%, less than 5%, or less than 1%.

The encapsulated phycocyanin in the pigment composition can have a high purity, as indicated by the A₆₂₀/A₂₈₀ ratio. In some embodiments, the encapsulated phycocyanin has a A₆₂₀/A₂₈₀ ratio of at least 0.7 (corresponding to food grade), at least 1, at least 1.5, at least 2, at least 2.5, at least 3, at least 3.5, or at least 4 (corresponding to analytical grade).

In another aspect, the present disclosure provides a food colorant comprising phycocyanin and milk fat globule membranes, wherein the phycocyanin is encapsulated in the milk fat globule membranes. For example, the phycocyanin encapsulated in the milk fat globule membranes can be produced by the encapsulation method as described here.

In yet another aspect, the present disclosure provides a pigment composition or a food colorant comprising the encapsulated phycocyanin product produced by the method as described herein.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms. As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter. As used herein, the singular forms “a”, “an”, and “the” include plural embodiments unless the context clearly dictates otherwise. The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9 to 1.1.

### EXAMPLES

### Example 1

### Phycocyanin Extraction

Cyanobacterial strains (Synechocystis sp. PCC 6803, Spirulina platensis UTEX LB2340, Synechococcus sp. PCC 7002, or Thermosynechococcus vestitus BP-1) were cultured to reach OD₇₃₀~4 (OD₇₃₀ stands for optical density at 730 nm and is a measure of culture density). The cultures were left in the dark for 24 hours to settle; the biomass settled at the bottom of the flask and the clear culture medium (~80%) was slowly poured off. Calcium chloride (CaCl₂)) was then added to the biomass to a final concentration of 0.5% w/v. The biomass was homogenized. For Synechocystis, homogenization was accomplished using a bead beater with four cycles of 45 s each in the presence of an equal volume of 0.1 mm glass beads. For Synechococcus and Thermosynechococcus, the bead beater was used with two cycles of 30 s each in presence of an equal volume of 0.1 mm glass beads. For Spirulina, three cycles of freezing and thawing (−20° C. for two hours, then thawing until thawed).

For each case, the homogenized slurry was then centrifuged (12,000×g for 15 minutes) at 4° C. The supernatant containing the phycocyanin was pipetted out, and the pellet was resuspended in 0.5% w/v CaCl₂) and centrifuged to extract any remaining phycocyanin. Supernatants were combined and centrifuged once again to remove any particulate matter. This yields food-grade phycocyanin (A₆₂₀/A₂₈₀ ratio of 0.7 or greater) that was stored at 4° C. until further purification.

A one-step ammonium sulfate precipitation was carried out by adding ammonium sulfate slowly to the phycocyanin extract while stirring gently, up to a concentration of 277 g/L (w/v) corresponding to 45% saturation at 25° C. for Synechocystis, Synechococcus and Thermosynechococcus. For Spirulina, ammonium sulfate was added to the phycocyanin extract in an amount to reach 50% saturation at 25° C. The mixture was incubated at 4° C. in the dark for 30 minutes while continuously stirring using a magnetic stirrer. Subsequently, the mixture was centrifuged at 4° C. (12,000×g for 15 min) and the supernatant was discarded. The phycocyanin precipitate was resuspended in water and centrifuged at 4° C. (12,000×g for 15 min) to remove any remaining cell debris and contaminants; the phycocyanin remained in the supernatant as the ammonium sulfate concentration had decreased. In order to remove any remaining ammonium sulfate, the extract was dialyzed against water. The A₆₂₀/A₂₈₀ ratio of the phycocyanin achieved at this stage was above 4, corresponding to analytical grade material.

The phycocyanin recovery rate achieved with this procedure was about 95% compared to what was originally present in the cells. It was demonstrated that this simple phycocyanin purification process works for all four strains of cyanobacteria that were tested, yielding a phycocyanin recovery rate of 90% or better, with the implication that this procedure may be applicable to most strains of cyanobacteria. Thus, a simple phycocyanin purification method was developed that is cost effective and works for a spectrum of cyanobacterial strains.

### Thermostability of Dry Phycocyanin Vs an Aqueous Solution of Phycocyanin

As an isolated, dry solid, phycocyanin demonstrates high thermostability when heated at 100° C. for 24 h. (FIG. 1, “Dry”). However, when the isolated phycocyanin is dissolved in water and heated at 50° C. for this same time period, the absorbance at 620 nm decreases, indicating degradation of the phycocyanin (FIG. 1, “Wet”).

### Phycocyanin Encapsulation

The method described here is for phycocyanin encapsulation in milk fat globule membranes (MFGMs). Another method that the inventors have shown to work well is encapsulation in phosphatidyl choline/cholesterol. Encapsulation in di-ether and tetra-ether lipids from hyperthermophilic archaea and bacteria is also promising.

For phycocyanin encapsulation the inventors extracted MFGMs from fresh cow milk according to reported procedure (Wang et al. 2022). The encapsulation process starts with dry phycocyanin powder that is resuspended in a polar solvent and dissolving MFGM in a nonpolar solvent; examples of solvents are indicated in Table 1. Phycocyanin can be suspended in polar solvents like methanol or dimethylformamide without apparent denaturation as the bright blue color is retained. The mixture (phycocyanin and lipids in two solvents) was mixed vigorously for five minutes and vortexed for another five minutes followed by drying in a speed vac until the solvents were evaporated completely.

TABLE 1
Combinations of solvents that have been used for
phycocyanin encapsulation into lipid vesicles.

| Identifier | Polar Solvent | Nonpolar Solvent |
|---|---|---|
| A | Dimethylformamide | Cyclohexane |
| B | Dimethylformamide | Hexanes |
| C | Dimethylformamide | Benzyl benzoate |
| D | Methanol | Ethyl acetate |
| E | Acetone | Ethyl acetate |
| F | Tetrahydrofuran | Methyl t-butyl ether |
| G | Acetonitrile | Cyclohexane |
| H | Methanol | Cyclohexane |

The dried pellet was resuspended in 50 mM sodium phosphate buffer (pH 7.5). Larger particles including encapsulated phycocyanin precipitated out because of the size of the particles. The phycocyanin precipitate was subjected to heat treatment (100° C. for 10 minutes). After heat treatment, a significant amount (typically more than half) of settled (presumably largely encapsulated) phycocyanin survived (i.e., did not bleach) and appeared to be normally colored under the microscope. These observations suggest that the dry phycocyanin particles were successfully protected from water by the lipid globules. In this case, minimal color loss was observed after phycocyanin encapsulation by MFGM in the presence of dimethylformamide and cyclohexane followed by heat treatment. This demonstrates that encapsulation of phycocyanin in lipids in the absence of water leads to thermostability of the pigment. Other solvent combinations are listed in Table 1. Further thermostability experiments are described in Example 2.

### Example 2

Encapsulation efficiency is aided by ionic charge on the surface of the phycocyanin and the encapsulant. This ionic charge can be modulated by pH. Specifically, lower pH can lead to more positive surface charges and higher pH can lead to more negative surface changes, while there is no net surface charge at the isoelectric point (pI). The pH values of the phycocyanin and MFGM suspensions, from which the phycocyanin and MFGM are isolated, can have an effect on the encapsulation efficiency when they are mixed in an organic solvent, as well as the interaction between protein and milk fat globule membranes. The effect of having a mild, net positive surface charge on the phycocyanin and a mild, net negative charge on the MFGM surface was studied.

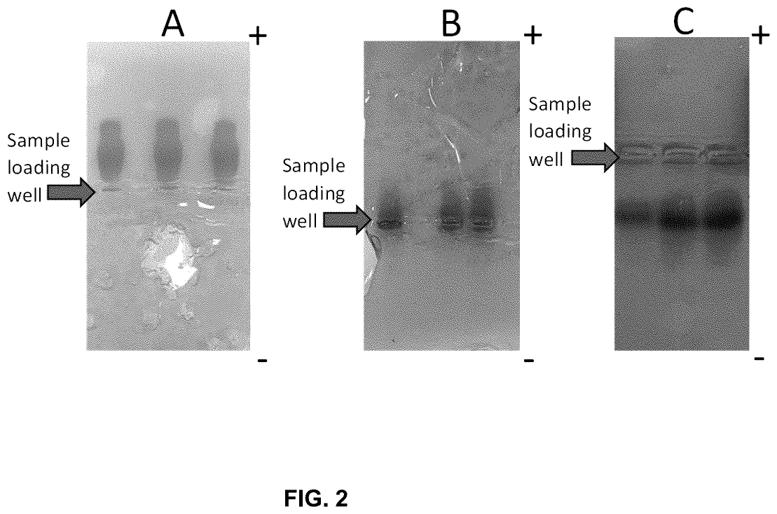

The pI of phycocyanin and milk fat globule membranes was experimentally determined by suspending the two components in buffers at various pH and observing whether they moved to the positive or the negative electrode in an agarose gel. Notably, the phycocyanin solution with a pH of 5.0 migrated to the positive electrode, the one at pH 4.5 went to the negative electrode, and the one at pH 4.8 migrated slightly towards the negative electrode, suggesting that pH 4.8 is close to its iso-electric point (no net surface charge at this pH) (FIG. 2). Therefore, suspension of phycocyanin at a pH lower than 4.8 will provide the phycocyanin with a net positive charge. Phycocyanin was suspended in buffer at pH 4.5 (net positive charge) or pH 4.8 (essentially neutral net surface charge) and the phycocyanin was freeze-dried. After freeze-drying, the samples were ground using a mortar and pestle and were then used for encapsulation.

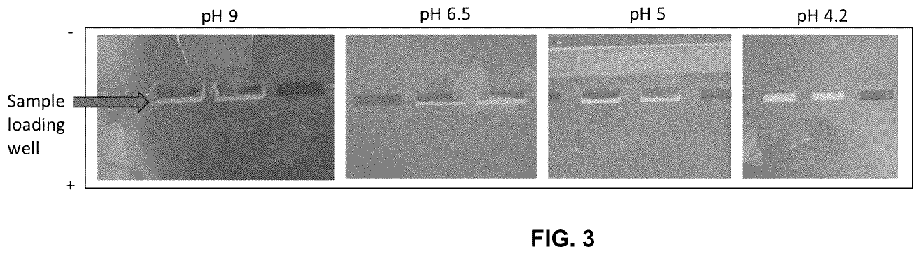

Similarly, milk fat globule membranes (MFGMs) were prepared and the pH of these MFGM extracts was adjusted. Unlike phycocyanin, MFGM is not a molecule or a complex of similar components, but rather it is a complex of different types of phospholipids, glycoproteins, and other biomolecules. It was found that, unlike phycocyanin, MFGM will not migrate much through an agarose gel due to its large size (FIG. 3). Thus, the inventors observed to which side of the loading well the MFGM moved when it was resuspended in sodium phosphate buffer (50 mM) at varying pHs and run in agarose gel electrophoresis buffer of the same pH to determine the net charge on the surface of the MFGMs depending on movement in the electric field.

As indicated in FIG. 3, MFGM suspensions with a pH of 9 or 6.5 showed a clear migration towards the positive electrode, at pH 5.0 MFGMs crowded a little less in the well toward the positive electrode, and at pH 4.2 we observed no movement of MFGM: at this pH the particles stayed dispersed in the loading well suggesting that pH 4.2 is close to its iso-electric point (no net surface charge at this pH). To provide MFGMs with a negative surface charge, we adjusted the pH of MFGM suspensions to 6.5 and subsequently freeze-dried the materials. After freeze-drying, the samples were ground using a mortar and pestle and were then used for encapsulation.

Six combinations of phycocyanin-to-MFGM ratios were evaluated (Table 2). For the encapsulation process, MFGMs were dried after pH adjustment to pH 6.5 and were subsequently suspended in 1 mL cyclohexane. Phycocyanin was also dried after pH adjustment (pH 4.8 or 4.5) and then suspended in 1 mL dimethylformamide (DMF). In total, six different combinations of phycocyanin and MFGM suspensions were prepared, as detailed in Table 2. For the control, the phycocyanin was prepared the same way except that no MFGM was added. The mixture (phycocyanin and MFGMs in two solvents) was mixed thoroughly for five minutes and vortexed for another five minutes followed by drying in a speed vac overnight. In addition, another control was used where the freshly extracted phycocyanin (without any treatments) was incubated in a boiling water bath (100° C.).

After the samples were dry, the mixture was resuspended in 50 mM sodium phosphate buffer (pH 7.5) and was centrifuged (5000×g for 5 minutes); the pellet was resuspended in 50 mM sodium phosphate buffer (pH 7.5). The resuspended pellet was incubated in a boiling water bath (100° C.) for different times and the absorbance spectrum of the sample was recorded.

TABLE 2
Different combinations of phycocyanin and MFGM used in this
study. In all combinations, the pH of MFGM was 6.5.

| phycocyanin pH | Sample | phycocyanin:MFGM ratio (mg/mg) |
|---|---|---|
| 4.8 | M1 | 3:1 |
| 4.8 | M2 | 6:1 |
| 4.8 | M3 | 12:1 |
| 4.5 | M4 | 3:1 |
| 4.5 | M5 | 6:1 |
| 4.5 | M6 | 12:1 |

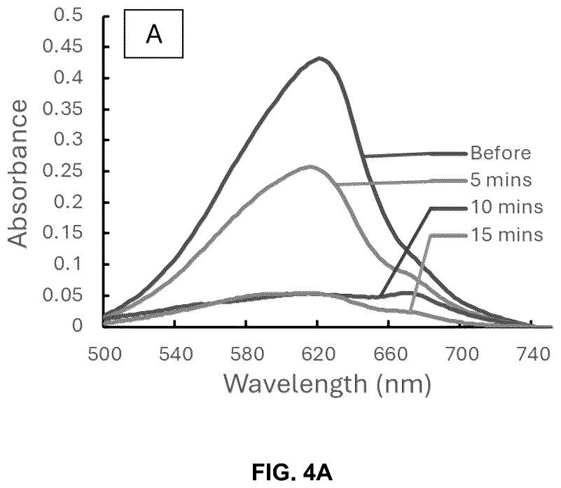

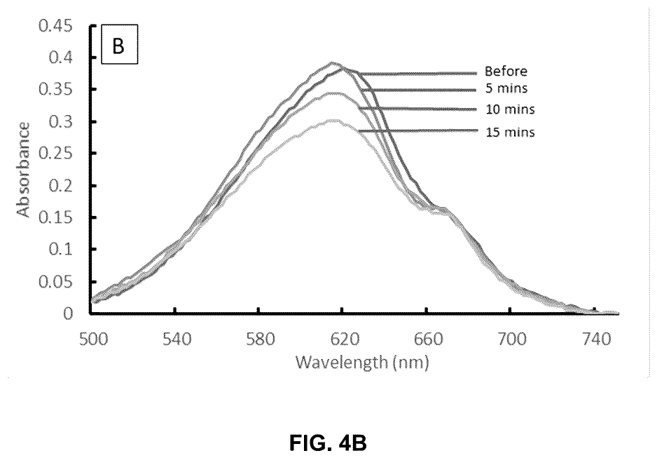

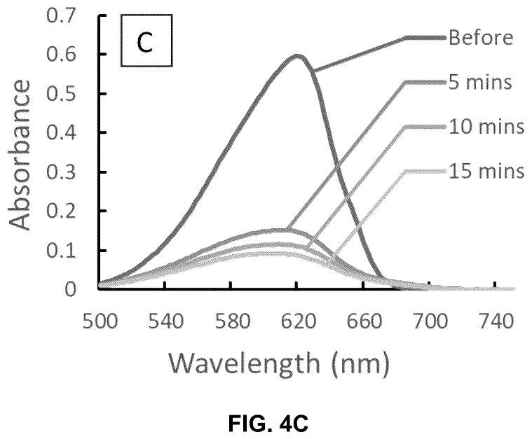

Table 3 and FIGS. 4A-4C show the results of this experiment. While the control (without MFGMs) and freshly isolated phycocyanin rapidly bleached out upon boiling (FIGS. 4A and 4C), after 5 min of boiling essentially no change in 620-nm absorbance was observed in the encapsulated sample M5 (FIG. 4B), thus illustrating significantly enhanced thermostability. Table 3 shows values for the percentage of 620-nm absorbance remaining after incubation at 100° C. for the times indicated compared to 620-nm absorbance before incubation at 100° C. The results indicate that 60-80% of A₆₂₀ (absorbance at 620 nm) was retained in all encapsulated samples after boiling for 15 min, with 80-100% of A₆₂₀ remaining after boiling for 5 min. In contrast, the unencapsulated control samples and freshly isolated phycocyanin were not thermostable and lost most of their A₆₂₀ within 5-10 min.

TABLE 3
Thermostability profile of different phycocyanin preparations.

| Sample | 5 mins | 10 mins | 15 mins |
|---|---|---|---|
| M1 | 99.9 | 89.6 | 77.2 |
| M2 | 83.8 | 69.7 | 60.1 |
| M3 | 88.8 | 70.2 | 66.9 |
| M4 | 90.7 | 80.9 | 78.4 |
| M5 | 104.2 | 85.2 | 73.8 |
| M6 | 87.1 | 81.9 | 69.7 |
| Control | 58.7 | 12.2 | 12.4 |
| Fresh phycocyanin | 24.6 | 18.5 | 14.6 |

### Example 3

Sucrose laurate has been investigated as a surfactant between phycocyanin and MFGMs. Here, 1.5 mg sucrose laurate was mixed in 1 mL cyclohexane and 12 mg phycocyanin was added. The mixture was mixed thoroughly and then vortexed for two minutes; the mixture was then dried in a speed-vac. In parallel, 0.6 mg MFGMs was suspended in cyclohexane and dried, creating a thin film on the walls of a 1.5 mL tube. DMF was added to the phycocyanin-sucrose laurate mixture and this solution was added to the 1.5 mL tube, containing MFGMs, and mixed. The mixture was again vortexed followed by drying in a speed-vac. This procedure also results in phycocyanin with improved thermostability vs. an unencapsulated control.

For reasons of completeness, various aspects of the disclosure are set out in the following numbered clauses:

Clause 1. A method of extracting phycocyanin, the method comprising:
adding a salt of a divalent metal ion to a slurry comprising phycocyanin to a final concentration of the salt of about 0.25% w/v to about 1% w/v;
homogenizing the slurry to form a homogenate;
centrifuging the homogenate to form a pellet and a supernatant comprising phycocyanin; and
isolating phycocyanin from the supernatant to form a phycocyanin extract.

Clause 2. The method of clause 1, wherein the divalent metal ion is Ca²⁺, optionally wherein the salt of the divalent metal ion is calcium chloride (CaCl₂)).

Clause 3. The method of any one of clauses 1-2, wherein isolating phycocyanin from the supernatant comprises:
isolating the supernatant from the pellet;
combining the isolated supernatant with ammonium sulfate to induce precipitation of the phycocyanin; and
isolating the precipitated phycocyanin to form the phycocyanin extract.

Clause 4. The method of clause 3, wherein isolating the supernatant from the pellet comprises:
resuspending the pellet in about 0.25% w/v to about 1% w/v CaCl₂) to form a suspension;
centrifuging the suspension to form an additional supernatant comprising phycocyanin; and
combining the supernatant with the additional supernatant to form the isolated supernatant.

Clause 5. The method of any one of clauses 3-4, wherein the ammonium sulfate solution is added to the isolated supernatant up to a concentration of about 277 g/L (w/v).

Clause 6. The method of any one of clauses 1-5, wherein the homogenization is carried out using a bead beater.

Clause 7. The method of any one of clauses 1-6, wherein the centrifugation is carried out at least at 12,000×g at 4° C.

Clause 8. The method of any one of clauses 1-7, wherein the slurry comprises cyanobacterial biomass, optionally wherein the cyanobacterial biomass is obtained from a cyanobacterial culture selected from the group consisting of Synechocystis sp. PCC 6803, Spirulina platensis UTEX LB2340, Synechococcus sp. PCC 11901, Synechococcus sp. PCC 7002, Thermosynechococcus vestitus BP-1, and a combination thereof.

Clause 9. The method of any one of clauses 1-8, further comprising drying the phycocyanin extract.

Clause 10. A phycocyanin extract produced by the method of any one of clauses 1-9.

Clause 11. A dried phycocyanin extract produced by the method of clause 9, optionally wherein the dried phycocyanin extract is a powder, optionally wherein the dried phycocyanin extract has a A₆₂₀/A₂₈₀ ratio of at least 0.7.

Clause 12 A method of encapsulating phycocyanin, the method comprising:
contacting a first liquid phase comprising phycocyanin and a polar solvent with an encapsulating agent comprising a lipid to form an encapsulation mixture, thereby the phycocyanin is encapsulated in the encapsulating agent,
wherein the first liquid phase is essentially free of water.

Clause 13. The method of clause 12, comprising:
combining the first liquid phase with a second liquid phase comprising the encapsulating agent and a nonpolar solvent to form the encapsulation mixture, thereby the phycocyanin is encapsulated in the encapsulating agent,
wherein the first liquid phase and the second liquid phase are essentially free of water.

Clause 14. The method of any one of clauses 12-13, wherein the first liquid phase is a suspension or solution prepared by mixing dry phycocyanin with the polar solvent.

Clause 15. The method of any one of clauses 13-14, wherein the second liquid phase is a solution of the encapsulating agent in the nonpolar solvent.

Clause 16. The method of any one of clauses 12-15, further comprising removing the polar solvent and the nonpolar solvent to form a dried encapsulation mixture.

Clause 17. The method of clause 16, further comprising resuspending the dried encapsulation mixture in a resuspending buffer to form a resuspended encapsulation mixture.

Clause 18. The method of clause 17, further comprising isolating encapsulated phycocyanin from the resuspended encapsulation mixture by precipitation, thereby producing an encapsulated phycocyanin product.

Clause 19. The method of clause 18, further comprising drying the encapsulated phycocyanin product.

Clause 20. The method of any one of clauses 17-19, wherein the resuspension buffer has a pH of about 3 to about 11.

Clause 21. The method of any one of clauses 12-20, wherein the encapsulating agent comprises a phospholipid, a di-ether lipid, a tetra-ether lipid, cholesterol, or a mixture thereof.

Clause 22. The method of clause 21, wherein the phospholipid comprises phosphatidylcholine (PC), phosphatidylethanolamine (PE), sphingomyelin (SM), or a mixture thereof.

Clause 23. The method of any one of clauses 12-22, wherein the encapsulating agent further comprises a protein, optionally wherein the protein is a membrane protein.

Clause 24. The method of any one of clauses 12-23, wherein the encapsulating agent comprises:
(a) milk fat globule membranes (MFGMs),
(b) phosphatidylcholine and cholesterol;
(c) a diether lipid;
(d) a tetra-ether lipid; or
(e) a mixture thereof.

Clause 25. The method of clause 24, wherein the encapsulating agent comprises milk fat globule membranes (MFGMs).

Clause 26. The method of any one of clauses 24-25, wherein the encapsulating agent comprises phosphatidylcholine and cholesterol.

Clause 27. The method of any one of clauses 12-26, wherein the polar solvent comprises methanol, acetone, tetrahydrofuran, dimethylformamide, acetonitrile, dichloromethane, or a mixture thereof.

Clause 28. The method of any one of clauses 13-27, wherein the nonpolar solvent comprises ethyl acetate, methyl t-butyl ether, cyclohexane, hexanes, benzyl benzoate, or a mixture thereof.

Clause 29. The method of any one of clauses 12-28, wherein, prior to encapsulation, the phycocyanin is treated to have a net positive surface charge and the encapsulating agent is treated to have a net negative surface charge.

Clause 30. The method of clause 29, wherein the first liquid phase is prepared by mixing dry phycocyanin with the polar solvent, and wherein the dry phycocyanin is prepared from drying a suspension of phycocyanin having a pH of about 4.5 to about 4.8.

Clause 31 The method of any one of clauses 29-30, wherein the encapsulating agent comprises milk fat globule membranes, and
wherein the milk fat globule membranes are prepared from drying a suspension of the milk fat globule membranes having a pH of about 6.5.

Clause 32. The method of any one of clauses 12-31, wherein the first liquid phase further comprises a surfactant.

Clause 33. The method of clause 32, wherein the surfactant is a fatty acid ester.

Clause 34. The method of any one of clauses 32-33, wherein the surfactant is sucrose laurate.

Clause 35. An encapsulated phycocyanin product produced by the method of clause 18.

Clause 36. A dried encapsulated phycocyanin product produced by the method of clause 19.

Clause 37. A pigment composition comprising phycocyanin encapsulated in an encapsulating agent, the encapsulating agent comprising a lipid.

Clause 38. The pigment composition of clause 37, wherein the encapsulating agent comprises milk fat globule membranes, a di-ether lipid, a tetra-ether lipid, cholesterol, a phospholipid, a glycolipid, a glycoprotein, a carbohydrate, or a mixture thereof;
optionally wherein the phospholipid comprises phosphatidylcholine (PC), phosphatidylethanolamine (PE), sphingomyelin (SM), or a mixture thereof.

Clause 39. The pigment composition of any one of clauses 37-38, wherein the encapsulated phycocyanin maintains color after a heat treatment of 100° C. for at least 5 min, as determined by a change in the absorbance at 620 nm of less than 20%.

Clause 40. The pigment composition of any one of clauses 37-39, wherein the encapsulated phycocyanin has a A₆₂₀/A₂₈₀ ratio of at least 0.7.

Clause 41. A food colorant comprising phycocyanin and milk fat globule membranes, wherein the phycocyanin is encapsulated in the milk fat globule membranes.

Clause 42. A pigment composition or a food colorant comprising the encapsulated phycocyanin product produced by the method of clause 18.

The previous description of the disclosed embodiments is provided to enable any person skilled in the art to make or use the invention. Various modifications to these embodiments will be readily apparent to those skilled in the art, and the generic principles defined herein may be applied to other embodiments without departing from the spirit or scope of the invention. Thus, the invention is not intended to be limited to the embodiments shown herein but is to be accorded the widest scope consistent with the principles and novel features disclosed herein.

We claim:

1. A method of extracting phycocyanin, the method comprising:
adding a salt of a divalent metal ion to a slurry comprising phycocyanin to a final concentration of the salt of about 0.25% w/v to about 1% w/v;
homogenizing the slurry to form a homogenate;
centrifuging the homogenate to form a pellet and a supernatant comprising phycocyanin; and
isolating phycocyanin from the supernatant to form a phycocyanin extract.

2. The method of claim 1, wherein the divalent metal ion is Ca2+, optionally wherein the salt of the divalent metal ion is calcium chloride (CaCl₂)).

3. The method of claim 1, wherein isolating phycocyanin from the supernatant comprises:
isolating the supernatant from the pellet;
combining the isolated supernatant with ammonium sulfate to induce precipitation of the phycocyanin; and
isolating the precipitated phycocyanin to form the phycocyanin extract.

4-7. (canceled)

8. The method of claim 1, wherein the slurry comprises cyanobacterial biomass, optionally wherein the cyanobacterial biomass is obtained from a cyanobacterial culture selected from the group consisting of Synechocystis sp. PCC 6803, Spirulina platensis UTEX LB2340, Synechococcus sp. PCC 11901, Synechococcus sp. PCC 7002, Thermosynechococcus vestitus BP-1, and a combination thereof.

9. The method of claim 1, further comprising drying the phycocyanin extract.

10. A phycocyanin extract produced by the method of claim 1.

11. A dried phycocyanin extract produced by the method of claim 9, optionally wherein the dried phycocyanin extract is a powder, optionally wherein the dried phycocyanin extract has a A₆₂₀/A₂₈₀ ratio of at least 0.7.

12. A method of encapsulating phycocyanin, the method comprising:
contacting a first liquid phase comprising phycocyanin and a polar solvent with an encapsulating agent comprising a lipid to form an encapsulation mixture, thereby the phycocyanin is encapsulated in the encapsulating agent,
wherein the first liquid phase is essentially free of water.

13. The method of claim 12, comprising:
combining the first liquid phase with a second liquid phase comprising the encapsulating agent and a nonpolar solvent to form the encapsulation mixture, thereby the phycocyanin is encapsulated in the encapsulating agent,
wherein the first liquid phase and the second liquid phase are essentially free of water.

14. The method of claim 12, wherein the first liquid phase is a suspension or solution prepared by mixing dry phycocyanin with the polar solvent.

15. The method of claim 13, wherein the second liquid phase is a solution of the encapsulating agent in the nonpolar solvent.

16. The method of claim 12, further comprising removing the polar solvent and the nonpolar solvent to form a dried encapsulation mixture.

17-20. (canceled)

21. The method of claim 12, wherein the encapsulating agent comprises a phospholipid, a di-ether lipid, a tetra-ether lipid, cholesterol, or a mixture thereof.

22. (canceled)

23. The method of claim 12, wherein the encapsulating agent further comprises a protein, optionally wherein the protein is a membrane protein.

24. The method of claim 12, wherein the encapsulating agent comprises:
(a) milk fat globule membranes (MFGMs),
(b) phosphatidylcholine and cholesterol;
(c) a diether lipid;
(d) a tetra-ether lipid; or
(e) a mixture thereof.

25-26. (canceled)

27. The method of claim 12, wherein the polar solvent comprises methanol, acetone, tetrahydrofuran, dimethylformamide, acetonitrile, dichloromethane, or a mixture thereof.

28. The method of claim 13, wherein the nonpolar solvent comprises ethyl acetate, methyl t-butyl ether, cyclohexane, hexanes, benzyl benzoate, or a mixture thereof.

29. The method of claim 12, wherein, prior to encapsulation, the phycocyanin is treated to have a net positive surface charge and the encapsulating agent is treated to have a net negative surface charge.

30-31. (canceled)

32. The method of claim 12, wherein the first liquid phase further comprises a surfactant.

33-36. (canceled)

37. A pigment composition comprising phycocyanin encapsulated in an encapsulating agent, the encapsulating agent comprising a lipid.

38-42. (canceled)
